(19) United States
(12) Patent Application Publication
(10) Pub. No.: US 2008/0044479 A1
(43) Pub. Date: Feb. 21, 2008

(54) Antimicrobial sanitizing formulations with skin protection properties

(76) Inventor: Kevin C. Stack, Miami Beach, FL (US)

Correspondence Address: MCHALE & SLAVIN, P.A., 2855 PGA BLVD, PALM BEACH GARDENS, FL, 33410, US

(21) Appl. No.: 11/839,227

(22) Filed: Aug. 15, 2007

Related U.S. Application Data
(60) Provisional application No. 60/822,476, filed on Aug. 15, 2006.

(51) Int. Cl. A61K 9/14; A61P 31/00

(52) U.S. Cl. 424/489

(57) ABSTRACT

The present invention is directed toward antimicrobial sanitizing formulations in the form of a medicated polymer/emulsion based product and the method by which it is produced. The products may be used as a topical antimicrobial and skin cleanser, which contain at least one antimicrobial agent in a base which forms a hydrophobic protective barrier, having persistent antimicrobial properties, upon application to the skin.

### CROSS REFERENCE TO RELATED APPLICATIONS

This application claims benefit of the filing date of U.S. Provisional Patent Application No. 60/822,476, filed on Aug. 15, 2006, the contents of which are herein incorporated by reference.

### FIELD OF THE INVENTION

This invention relates to sanitizing formulations having antimicrobial properties; and particularly to highly persistent antimicrobial sanitizing and skin care products which display unique barrier properties.

### BACKGROUND OF THE INVENTION

Hand washing has long been recognized as a particularly effective method for reducing the transmission of communicable diseases. In hospitals, where patients are in a weakened condition, it is most important for health-care professionals to utilize an antimicrobial hand cleaning composition to prevent the spread of various pathogenic microorganisms. Furthermore, it is necessary to treat parts of the skin and mucous membranes antiseptically prior to any type of surgical procedure, injection, or puncture so as to prevent the transmission of infectious microorganisms. In such environments, compositions such as alcohols are effective antimicrobials. However, the defatting properties of alcohols cause chapping and cracking to occur to the skin of the user. The resultant damaged skin is then more prone to additional infectious contamination, since pathogenic microorganisms can enter and evade sanitizing materials by residing within the cracked epidermal layer. Additionally, the presence of alcohols inhibits the foaming action of various detergent compositions which are likely to be used in combination therewith. Various antimicrobials are known for use in such formulations, for example, iodophors, iodine formulations, phenolic compounds, e.g. hexachlorophene, and bisbiguanides. Such antimicrobial ingredients are also well-known additives for a variety of products, such as deodorant soap bars, underarm deodorants, liquid soaps and fabric treatments.

In order to form an efficacious antimicrobial product which is not injurious to the user's skin, various proposals have been made. Improvements in mildness and skin after-feel have called for the addition of such additives as glycerin, sorbitol, vitamin E, coco fatty acid derivatives and their salts, and sugar esters.

### DESCRIPTION OF THE PRIOR ART

U.S. Pat. No. 5,173,216 discloses a composition for decontaminating and/or disinfecting the hands comprising an amphoteric-cationic surfactant, a cationic surfactant, a wetting agent which is compatible with the cationic surfactant, and a nonionic regreasing agent. The composition exhibits both bacteriostatic and fungistatic effectiveness at varying concentrations.

U.S. Pat. No. 5,259,984 discloses a cleansing composition containing a storage-stable volatile polymer gel solution and a cleaning agent including an alkali metal hydroxide. In a preferred embodiment, the polymer gel solution includes a hydroxypropylmethylcellulose polymer. The composition is formed by forming a pre-mixed cleaning agent and a pre-mixed volatile aqueous gel solution. These pre-mixed components are then intermixed to form the final cleaner composition.

U.S. Pat. No. 5,562,912 discloses a cleansing composition containing an EO/PO/EO tri-block nonionic copolymer surfactant in conjunction with a generic skin cleanser composition.

U.S. Pat. No. 5,591,442 is drawn to an antiseptic and disinfectant hand cleaning composition containing a synergistic mixture of an alkyl alcohol component and a glycerol monoalkyl ether.

U.S. Pat. No. 5,650,143 drawn to a deodorant cosmetic stick composition provides a deodorant cosmetic stick product which has a translucent or transparent light transmitting appearance. The cosmetic stick contains propylene glycol, sodium stearate, dimethicone copolyol, TRICLOSAN, PENTADOXYNOL-200, and water.

U.S. Pat. No. 5,629,006 discloses a cleansing composition containing an alcohol, a block copolymer, a foaming surfactant, an emulsifier, a cleaning agent, a polyalkylene glycol, an emollient and water. Stepwise addition of the components with continuous mixing to a point of homogeneity is utilized in the method of formulation.

U.S. Pat. No. 5,719,113 discloses an antimicrobial cleansing composition containing chlorohexidine, a nonionic surfactant which does not include polyoxypropylene/polyoxyethylene block copolymers, an amphoteric surfactant, and an alkyl polyglucoside. Additionally included are viscosifiers or thickeners, emollients, fragrances, perfumes, coloring agents, preservatives, foaming agents, vitamins and fungicides.

U.S. Pat. No. 5,728,662 discloses a cleansing composition which consists essentially of a d-limonene, a solvent, a C₁₁ alcohol ethoxylate, polyoxyethylene (20) sorbitan monooleate, a water-soluble acrylic polymer, sodium hydroxide, mixed isothioazolinones, 2,6-di-tert-butyl-p-cresol and water.

U.S. Pat. No. 5,750,579 is drawn to a cleansing composition which is useful for the hands and fingers. The composition is in the form of a solution which comprises a disinfecting medicament in an alcohol and a thickening agent consisting of a combination of a carboxyvinyl polymer and a water-soluble, high molecular weight cellulose compound. The process of manufacture requires that various of the ingredients are blended to a point of homogeneity, resulting in a final, homogeneous composition.

U.S. Pat. No. 5,767,163 discloses a cleansing composition and method for its use as a hand antiseptic. The composition is an alcoholic solution containing cetyl alcohol, glycolic acid, benzalkonium chloride and isopropyl alcohol as its major constituent.

U.S. Pat. No. 5,772,640 drawn to TRICLOSAN-containing medical devices, discloses polymeric medical articles containing the anti-infective agents chlorohexidine and TRICLOSAN. The patent discloses a synergistic relationship between these compounds which permits the use of relatively low levels of both agents, while achieving effective antimicrobial activity when these compounds are contained in either hydrophilic or hydrophobic polymers.

Theses aforementioned prior art formulations suffer from the fact that increased use of various surfactants and lipid-restoring compositions reduce the effectiveness of the antimicrobial active ingredient.

U.S. Pat. Nos. 6,187,327 and 6,517,854, both to the present inventor, disclose an antimicrobial hand sanitizing lotion in the form of a medicated polymer/emulsion-based product and the method by which it is produced. The product is intended to be used as a topical antimicrobial and skin protective lotion and contains 2,4,4′-trichloro-2′-hydroxydiphenyl ether (available under the tradename TRICLOSAN or IRGASAN DP 300 from the Ciba Geigy Corp.) as the antimicrobial agent of choice in a base which forms a hydrophobic protective barrier, having persistent antimicrobial properties, upon application to the skin. While these patents have satisfied a long-felt need in the art there still exists a need for more skin sanitizing formulations that do not reduce the effectiveness of the antimicrobial active ingredients therein.

### SUMMARY OF THE INVENTION

The present invention describes antimicrobial sanitizing formulations in the form of a medicated polymer/emulsion based product and the method by which it is produced. The products may be used as a topical antimicrobial lotion, wipe, soap or related skin-cleansers. TRICLOSAN, Chloroxylenol, and quaternary ammonium compounds such as benzalkonium and benzethonium types of agents (e.g., benzalkonium chloride, and benzethonium chloride) are the antimicrobial agents of choice in the present formulations. For example, TRICLOSAN has demonstrated efficacy against the gram-positive and gram-negative bacteria, plus fungi and yeasts (see list in U.S. Pat. Nos. 6,187,327 and 6,517,854, herein incorporated by reference.)

It has been discovered that incorporation of at least one of the aforementioned antimicrobial agents in a formulation comprised of a Surfactant Phase, and a Wax Phase result in a product which is particularly effective in preventing cross-contamination of pathogenic microorganisms in the workplace. For example, TRICLOSAN is persistent in that it significantly reduces the incidence of bacteria on skin surfaces for a period of about 3-4 hours. It is applicable to any area of intact skin, and will kill pathogenic bacteria on contact and remain effective for extended periods of time. The mechanism of microbicidal action of these antibacterial agents is thought to be due to the disruption of intermolecular actions of the microbes.

The formulations of the present invention are non-toxic and hypoallergenic. These formulations provide at least one broad spectrum anti microbial which forms a polymeric film on healthy skin and create a safe and long-lasting product that will not rub off due to its unique bonding agent. The hydrophobic portion of the process utilizes a USP White Wax in combination with the acrylic carbomer. The wax in solution in co-ordination with the product backbone (CARBOPOL 934-P), melts through the heat of the hand. The wax phase spreads over the skin with the CARBOPOL theorized to act in two ways. The acrylate chains are theorized to intercalate into the wax matrix and stabilize the wax by adding support to the horizontal spreading and layering of the wax. Further, the CARBOPOL is believed to interact with the skin surface relative to the horizontal wax layer. The combination of these interactions forms a physical hydrophobic layer which resides on the skin surface and provides a barrier which would inhibit penetration of liquids which are primarily hydrophilic in nature. The wax is solubilized and dispersed with the aid of surfactants and dimethicone within an alcohol/glycerol base. Stearic acid, particularly triple pressed, is noted as being critical to affecting complete solubilization of the raw materials in the wax phase. The formulations include at least one antimicrobial ingredient at appropriate concentration ranges resulting in a product that is efficacious for use, especially by healthcare professionals.

One of the unique properties of the product is its ability to protect the skin from relatively strong acids and bases. Tests conducted on metallic surfaces demonstrated enhanced longevity of the metallic substrates when exposed to corrosive environments. The barrier properties of the instant composition further increase the efficiency of bacterial removal from the skin's surface. The product is further characterized by exhibiting a highly persistent antimicrobial action. This persistence may be attributed to the stability of the wax/carbomer hydrophobic layer which allows for a unique physical presentation of the antimicrobial. The stabilized barrier composition is stabilized by the CARBOPOL chains orientated into the wax phase. For example, consider TRICLOSAN which is a hydrophobic molecule. The hydrophobic molecule will orientate itself with respect to the barrier layer, resulting in a product which maintains persistent skin contact and antimicrobial action. In combination, these properties result in a product having enhanced effectiveness in the removal of surface bacteria compared to soap that simply rinses off. This effectiveness persists for the duration of the presence of the product formulation on the skin. Application of this product prior to a soap and water hand washing has been clinically proven to enhance hand washing with a statistically significant increase in the removal of harmful bacteria from the skin surface, compared to ordinary hand washing without prior application of the product.

When used in combination with latex gloves, the product inhibits the growth of microorganisms underneath the latex gloves, protects hands from contamination should the gloves become damaged, moisturizes and soothes the skin to combat the potential damaging effects of latex, harsh soaps and frequent washing.

When processing the formulations of the present invention, the surfactant and wax phases are each formulated according to particular concentration and processing parameters, and then blended to form a Final Phase, resulting in unique topical antimicrobial sanitizing and skin care products.

Accordingly, it is an objective of the instant invention to teach various antimicrobial sanitizing formulations, especially effective as a skin sanitizer, which is efficacious for a broad range of microorganisms and is characterized by unique skin protective barrier properties and enhanced persistence.

It is a further objective of the instant invention to teach a method for producing sanitizing formulations wherein adherence to particular process parameters results in a unique final product.

It is yet another objective of the instant invention to teach skin protective and sanitizing formulations wherein contact with the skin results in destruction of microbial contaminants and simultaneous formation of a hydrophobic skin protective surface layer.

It is a still further objective of the invention teach a skin protective and sanitizing formulation that can be used as a lotion, wipe, encapsulated beads, soap or related skin-cleansers.

These and other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### DETAILED DESCRIPTION OF THE INVENTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

In accordance with the present invention acrylic acid polymers which can be used in the vehicle disclosed herein include any nontoxic charged water soluble polymer. For example, high molecular weight, crosslinked copolymers of acrylic acid and C10-C30 alkyl acrylate, such as polymers sold under the tradename PEMULEN, CARBOPOL polymers, polymeric emulsifiers and NOVEON polycarbophils, which are polymers of acrylic acid, crosslinked with polyalkenyl ethers or divinyl glycol. These polymers can either be negatively or positively charged. Typically, negatively charged polymers will include, but are not limited to, carboxy vinyl polymers, such as CARBOPOL® 934P NF polymer which can be used to formulate thick gels, emulsions and suspensions.

Surfactants useful in accordance with the present invention include the TRITON X Series of surfactants, which are versatile nonionic surfactants recognized for their wetting, detergency, superior hard surface, metal cleaning and excellent emulsification performance. TRITON X Series surfactants are used in almost every type of liquid, paste, and powdered cleaning compound, ranging from heavy-duty industrial products to gentle detergents. They are important ingredients of primary emulsifier mixtures used in the manufacture of emulsion polymers and stabilizers in latex polymers. TRITON X Series surfactants are recognized for pigment wetting and stabilization in coatings, and are offered in a range of HLB to match specific wetting and dispersing requirements. They are referred to under the names Alkylaryl polyether alcohol; Octyl phenol ethoxylate; Triton X-100 Surfactant; Polyoxyethylated octyl phenol; and alpha-[4-(1,1,3,3-tetramethylbutyl)phenyl]-omega-hydroxypoly(oxy-1,2-ethanediyl);

Chelating agents useful in accordance with the present invention include compounds sold under the tradename VERSENE* 100, which is a chelating agent provided as an aqueous solution of the tetrasodium salt of ethylenediaminetetraacetic acid. Na4EDTA is the strongest, most versatile, and widely used chelant for controlling metal ions over a broad pH range in aqueous systems.

Nonionic surfactants, such as TERGITOL NP Series nonionic surfactants deliver a combination of economy and performance in a wide variety of applications, including cleaning product formulations, paints and coatings, emulsion polymerization, and many others. These NPE surfactants are used anywhere there is a need for increased surface activity, and provide excellent all-purpose detergency and wetting, as well as solubilization and emulsification. Nonylphenol Ethoxylates (NPE)

Production of the antimicrobial sanitizing formulations of the present invention relies upon adherence to a particular set of process parameters in order to arrive at a unique final product. Additionally, it is necessary that rigorous homogenization be carried out to form a “grain” free product. Finally, the various steps must be carried out within particular temperature ranges which are critical to the outcome of the process.

Excipients useful in forming the antimicrobial and skin protective formulations (lotions, wipes, soaps, encapsulated beads, or the like) according to various embodiments of the present invention are described in the following examples.

### EXAMPLE 1

In formulating a batch of the antimicrobial sanitizing and skin protective lotion according to one embodiment of the invention, excipients useful in the manufacture of this product were added in the following approximate amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | TRITON X-100 | 1.0-8.0 |
| (5) | PROPYLENE GLYCOL U.S.P. | 0.5-5.0 |
| (6) | TERGITOL NP-9 | 1.0-8.0 |
| (7) | DOWCIDE-A | 0.0-2.0 |
| (8) | TRICLOSAN | 0.1-0.8 |
| (9) | TRIETHANOLAMINE 85% N.F | 0.5-2.0 |
| (10) | CHLOROHEXIDINE DIGLUCONATE 20% | 0.1-7.0 |
| (11) | ALPHA TOCOPHEROL | 0.5-8.0 |

Wax Phase

| | | |
|---|---|---|
| (12) | STEARIC ACID - TRIPLE PRESSED | 2.0-4.0 |
| (13) | CETYL ALCOHOL N.F. | 0.75-2.0 |
| (14) | ETHYLENE GLYCOL MONOSTEARATE | 0.25-1.5 |
| (15) | DIMETHICONE L-45-350 CSTKS | 0.5-3.5 |
| (16) | USP WHITE WAX | 0.1-1.0 |
| (17) | PARAGON MEPB | 0.5-6.0 |

The following method steps were followed:

Step (A) The first part of the Surfactant Phase is formulated by combining the appropriate amounts of following ingredients:

| | |
|---|---|
| 1) | Deionized Water of reagent grade |
| | exhibiting less than 1 micro-ohm resistivity is first added |
| | to a mixing tank; |
| 2) | VERSENE 100 (or a like equivalent EDTA Sodium Salt); |
| 3) | CARBOPOL 934 P(or a like equivalent Acrylic Polymer). |

The mixer is engaged in the reverse mode while the circulating pump is turned on to full open, yielding a flow rate of about 110-150 gallons/minute (gpm) at a pressure of about 60-110 psi, for recirculation of the mixture. Engagement of the pump in the reverse mode causes mixing to occur in a bottom to top direction within the tank. This reverse mode pumping coupled with the forceful agitation of the recirculating pump is critical in solubilizing the CARBOPOL-934 in the mixture.

Homogenization of the above-mentioned ingredients is then carried out for about 30-40 minutes utilizing a stator-bladed motor driven homogenizer under flow conditions of about 110-150 gpm and at a pressure of about 60-110 psi, which conditions are sufficiently rigorous to yield a “grain” free and highly uniform product.

The remaining excipients in the Surfactant phase are weighed and added to the mixture:

| | |
|---|---|
| 4) | TRITON X-100 Surfactant (or a like equivalent Octyl |
| | Phenyoxypolyethoxy non-ionic surfactant); |
| 5) | Propylene Glycol (USP); |
| 6) | TERGITOL NP-9 Surfactant (or a like equivalent |
| | Nonylphenol polyethylene glycol ether non-ionic surfactant); |
| 7) | DOWCIDE-A (or a like equivalent Sodium O- |
| | Phenylphenatetetrahydrate); |
| 8) | TRICLOSAN (2,4,4′-trichloro-2′-hydroxydiphenyl ether); |
| 9) | Triethanolamine 85% N.F.; |
| 10) | Chlorohexidine Digluconate 20% (CHG); |
| 11) | Alpha Tocopherol. |

It is noted that the hydrophilic portion of the product is modified by the use of the non-ionic surfactant (TRITON X-100) in a propylene glycol base. The hydrophilic phase is further modified due to the inclusion of TERGITOL NP-9 which includes the nonoxyl class of compounds.

Inclusion of Alpha Tocopherol (Alpha Tocopherol Acetate) commonly known as Vitamin E has a two-fold benefit. Its presence inhibits oxidation of the product as well as providing additional skin conditioning properties. Since tocopherols are freely soluble in alcohols and lipids, they easily penetrate the skin layer and provide conditioning benefits.

After all ingredients have been blended, the Surfactant Phase is then heated to within a range of about 70° C.-85° C., and maintained within this temperature range while mixing and pump recirculation are continued at about 110-150 gpm at a pressure of about 60-110 psi.

Step (B) The Wax Phase is next formulated by adding the appropriate amounts of the following ingredients except PARAGON:

| | |
|---|---|
| (12) | Stearic Acid - Triple Pressed; |
| (13) | Cetyl Alcohol N.F.; |
| (14) | Ethylene Glycol Monostearate; |
| (15) | Dimethicone L-45-350 cstks; |
| (16) | White Wax (BARECO BE SQUARE). |

These ingredients are heated to within a range of about 70° C.-85° C., ideally about 77° C.-80° C.; and maintaining the temperature of the Wax Phase within this temperature range, while mixing at about 1500-1700 revolutions/minute (rpm) using a direct drive mixer.

The use of a wax, e.g. BARECO BE SQUARE, or a like equivalent which is a USP grade White Wax having a melting point in the range of 70° C.-85° C., provides a unique property. The wax, which is in solution in coordination with the CARBOPOL-934, melts through contact with the heat of the hands. This in turn forms a physical hydrophobic layer and provides a barrier which appears to inhibit penetration of liquids which are primarily hydrophilic in nature. This property helps protect the user from injury due to contact injurious materials, e.g. with acids and/or bases. The wax is apparently solubilized and dispersed with the aid of the surfactants and Dimethicone within an alcohol/glycerol base. The presence of Stearic acid, particularly triple pressed, is critical to effecting the complete solubilization of the remaining Wax Phase materials.

While not wishing to be bound to any particular theory, it is believed that the wax flattens to form a neutral and hydrophobic barrier. The carbomers are believed to support the wax layer in the horizontal plane and in attachment to the skin. The carbomer molecule, which is believed to physically intercalate within the wax phase, thereby reinforcing the wax layer, is also believed to interact with the skin thereby having a stabilizing effect upon the wax layer, which results in the enhanced persistence characteristic of the product. Lastly, it is believed that the processing steps orient the TRICLOSAN molecules to yield an optimum level of antimicrobial activity.

Step (c) The Final Phase is formed by adding the Wax Phase to the Surfactant Phase.

At the time of mixing, the Wax Phase is being maintained at approximately 85° C. and the Surfactant Phase is maintained at 80° C. The mixing takes place by using homogenization, recirculation and pressure. Pressure generation is accomplished by restricting the outlet side of the pump, thus limiting the flow therethrough. This restriction keeps the pump stators full at all times, so as to avoid burn out of the pump. Such conditions are maintained for 45-60 minutes using a 20 HP pump, at a rate of about 100-150 gpm, at about 60-110 psi, in reverse mode, restricting the outlet and recirculating the batch. After approximately 60 minutes, the temperature is then lowered to less than 50° C. so that the PARAGON MEPB Parabens materials can be safely added.

Step (D) PARAGON MEPB (a mixture of Methyl, Ethyl, Propyl, and Butyl Parabenzene in a Phenoxy Ethanol solvent, or a like equivalent mixture) is then added and homogenization is continued for an additional 20-30 minutes with the recirculation pump on full open. In a particular embodiment, the MEPB mixture had about 16% methyl paraben, about 4% ethyl paraben, about 2% propyl paraben, about 6% butyl paraben and the remainder, about 72% of phenoxy-ethanol solvent.

It is theorized that inclusion of DOWCIDE-A, Chlorohexidine Digluconate (CHG) and the Parabens species in a Phenoxy-Ethanol solvent act as phenolic based preservatives to further increase hydrophobic solubility and thereby potentiate the active biocidal properties of the product.

It is further theorized that the propylene glycol, cetyl alcohol, phenoxyethyl alcohol, parabens, and octyl phenol act as permeability barriers to the bacterial lipid cell wall; that the TRITON-X 100 and triethanolamine offer an ionic approach to cell wall disruption via a chelation mechanism; and that the phenoxyethyl alcohol, parabens and DOWCIDE-A further provide cytoplasmic membrane permeation.

### EXAMPLE 2

The following formulation is a lotion similar to Example 1 above. However, this example does include titanium dioxide, an essential oil package, and farnesol. The addition of titanium dioxide at the lower levels acts a whitening pigmentation for aesthetic purposes and at higher levels titanium dioxide will provide sun blocking protection to the user. The essential oil package is an emollient that provides enhanced skin conditioning properties to the user. The farnesol ingredient is an organic compound which is believed to act as a natural preservative.

EOP-122 is based on: Tocopheryl Acetate (d-Alpha, Natural) 38.50%; Grape Seed Oil (Ultra Pure Grade, Non-Scented) 28.5%; Avocado Oil (Ultra Pure Grade, Non-Scented) 21.00%; Jojoba Oil (Ultra Pure Grade, Non-Scented) 9.50%; Triton X-100, 2.50%.

Excipients useful in the manufacture of this product were added in the following approximate amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | TRITON X-100 | 1.0-8.0 |
| (5) | TITANIUM DIOXIDE | 0.05-7.0 |
| (6) | FARNESOL | 0.05-5.0 |
| (7) | ESSENTIAL OIL PACKAGE (#EOB-122) | 0.5-8.0 |
| (8) | PROPYLENE GLYCOL U.S.P. | 0.5-5.0 |
| (9) | TERGITOL NP-9 | 1.0-8.0 |
| (10) | DOWCIDE-A | 0.0-2.0 |
| (11) | TRICLOSAN | 0.10-0.8 |
| (12) | TRIETHANOLAMINE 85% N.F | 0.5-2.0 |
| (13) | CHLOROHEXIDINE DIGLUCONATE 20% | 0.1-7.0 |
| (14) | ALPHA TOCOPHEROL | 0.5-8.0 |

Wax phase

| | | |
|---|---|---|
| (15) | STEARIC ACID - TRIPLE PRESSED | 2.0-4.0 |
| (16) | CETYL ALCOHOL N.F. | 0.75-2.0 |
| (17) | ETHYLENE GLYCOL MONOSTEARATE | 0.25-1.5 |
| (18) | DIMETHICONE L-45-350 CSTKS | 0.5-3.5 |
| (19) | USP WHITE WAX | 0.1-1.0 |
| (20) | PARAGON MEPB | 0.5-6.0 |

The method for making the formulation in Example 2 follows the same steps A-D outlined in Example 1 above, the only difference is the addition of titanium dioxide, essential oil package, and farnesol into the surfactant phase of Step B.

### EXAMPLE 3

The following formulation is similar to Example 1 above but without the non-ionic surfactants TRITON X-100 and TERGITOL NP-9 as these surfactants have the potential for irritation, sensitization and allergic response on the skin of some users. In addition, this example includes titanium dioxide, essential oil package and farnesol.

Excipients useful in the manufacture of this product were added in the following approximate amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | PROPYLENE GLYCOL U.S.P. | 0.5-5.0 |
| (5) | DOWCIDE-A | 0.0-2.0 |
| (6) | TRICLOSAN | 0.10-0.8 |
| (7) | TRIETHANOLAMINE 85% N.F | 0.5-2.0 |
| (8) | CHLOROHEXIDINE DIGLUCONATE 20% | 0.1-7.0 |
| (9) | ESSENTIAL OIL PACKAGE (#EOP-122) | 0.5-8.0 |
| (10) | TITANIUM DIOXIDE | 0.05-7.0 |
| (11) | FARNESOL | 0.05-5.0 |
| (12) | ALPHA TOCOPHEROL | 0.5-8.0 |

Wax Phase

| | | |
|---|---|---|
| (13) | STEARIC ACID-TRIPLE PRESSED | 2.0-4.0 |
| (14) | CETYL ALCOHOL N.F. | 0.75-2.0 |
| (15) | ETHYLENE GLYCOL MONOSTEARATE | 0.25-1.5 |
| (16) | DIMETHICONE L-45-350 CSTKS | 0.5-3.5 |
| (17) | USP WHITE WAX | 0.1-1.0 |
| (18) | PARAGON MEPB | 0.5-6.0 |

The method for making the formulation in Example 3 follows the same steps A-D outlined in Example 1 above, the only difference is that TRITON X-100 and TERGITOL NP-9 are not included at step B and titanium dioxide, essential oil package, and farnesol are.

### EXAMPLE 4

The following lotion formulation is similar to Example 3 in that it does not include surfactants TRITON X-100 and TERGITOL NP-9; however, the formulation does include TWEEN-20 although is contemplated herein that any other sorbitol based surfactant or equivalent could be used without departing from the scope of the invention. As with the previous examples, this formation includes titanium dioxide, essential oil package and farnesol.

Excipients useful in the manufacture of this product were added in the following approximate amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | TWEEN-20 | 0.5-8.0 |
| (5) | PROPYLENE GLYCOL U.S.P. | 0.5-5.0 |
| (6) | DOWCIDE-A | 0.0-2.0 |
| (7) | TRICLOSAN | 0.10-0.8 |
| (8) | TRIETHANOLAMINE 85% N.F | 0.5-2.0 |
| (9) | CHLOROHEXIDINE DICLUCONATE 20% | 0.1-7.0 |
| (10) | ALPHA TOCOPHEROL | 0.5-8.0 |
| (11) | ESSENTIAL OIL PACKAGE (#EOP-122) | 0.5-8.0 |
| (12) | TITANIUM DIOXIDE | 0.05-7.0 |
| (13) | FARNESOL | 0.05-5.0 |

Wax phase

| | | |
|---|---|---|
| (14) | STEARIC ACID - TRIPLE PRESSED | 2.0-4.0 |
| (15) | CETYL ALCOHOL N.F. | 0.75-2.0 |
| (16) | ETHYLENE GLYCOL MONOSTEARATE | 0.25-1.5 |
| (17) | DIMETHICONE L-45-350 CSTKS | 0.5-3.5 |
| (18) | USP WHITE WAX | 0.1-1.0 |
| (19) | PARAGON MEPB | 0.5-6.0 |

The method for making the formulation in Example 4 follows the same steps A-D outlined in Example 1 above, the only difference is that TRITON X-100 and TERGITOL NP-9 are not included at step B and titanium dioxide, essential oil package, farnesol and TWEEN-20 are included in step B.

### EXAMPLE 5

The following lotion is similar to Example 4 above in that it does not include surfactants TRITON X-100 and TERGITOL NP-9, but it does include the surfactant TWEEN-20 or any other suitable sorbitol-based surfactant or equivalent. In addition, this example includes titanium dioxide, essential oil package and farnesol. This formulation does not include the antimicrobial agents TRICLOSAN or Chlorohexidine Digluconate (CHG).

Excipients useful in the manufacture of this product were added in the following approximate amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | TWEEN-20 | 0.5-8.0 |
| (5) | PROPYLENE CLYCOL U.S.P. | 0.5-5.0 |
| (6) | DOWCIDE-A | 0.0-2.0 |
| (7) | TRIETHANOLAMINE 85% N.F | 0.5-2.0 |
| (8) | ESSENTIAL OIL PACKAGE (#EOP-122) | 0.5-8.0 |
| (9) | ALPHA TOCOPHEROL | 0.5-8.0 |
| (10) | TITANIUM DIOXIDE | 0.05-7.0 |
| (11) | FARNESOL | 0.05-5.0 |

Wax phase

| | | |
|---|---|---|
| (12) | STEARIC ACID - TRIPLE PRESSED | 2.0-4.0 |
| (13) | CETYL ALCOHOL N.F. | 0.75-2.0 |
| (14) | ETHYLENE GLYCOL MONOSTEARATE | 0.25-1.5 |
| (15) | DIMETHICONE L-45-350 CSTKS | 0.5-3.5 |
| (16) | USP WHITE WAX | 0.1-1.0 |
| (17) | PARAGON MEPB | 0.5-6.0 |

The method for making the formulation in Example 5 follows the same steps A-D outlined in Example 1 above, the only difference is that TRITON X-100, TERGITOL NP-9, TRICLOSAN, and Chlorohexidine Digluconate (CHG) are not included at step B and titanium dioxide, essential oil package, farnesol and TWEEN-20 are included in step B.

### EXAMPLE 6

The following lotion does not include any surfactants. Moreover, this formulation does not include the antimicrobial agents TRICLOSAN or Chlorohexidine Digluconate (CHG). The formulation of this example does include titanium dioxide, essential oil package, farnesol.

Excipients useful in the manufacture of this product were added in the following approximate amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | PROPYLENE GLYCOL U.S.P. | 0.5-5.0 |
| (5) | DOWCIDE-A | 0.0-2.0 |
| (6) | TRIETHANOLAMINE 85% N.F | 0.5-2.0 |
| (7) | TITANIUM DIOXIDE | 0.05-7.0 |
| (8) | FARNESOL | 0.05-5.0 |
| (9) | ESSENTIAL OIL PACKAGE (#EOP-122) | 0.5-8.0 |
| (10) | ALPHA TOCOPHEROL | 0.5-8.0 |

Wax Phase

| | | |
|---|---|---|
| (11) | STEARIC ACID - TRIPLE PRESSED | 2.0-4.0 |
| (12) | CETYL ALCOHOL N.F. | 0.75-2.0 |
| (13) | ETHYLENE GLYCOL MONOSTEARATE | 0.25-1.5 |
| (14) | DIMETHICONE L-45-350 CSTKS | 0.5-3.5 |
| (15) | USP WHITE WAX | 0.1-1.0 |
| (16) | PARAGON MEPB | 0.5-6.0 |

The method for making the formulation of Example 6 follows the same steps A-D outlined in Example 1 above, the only difference is that TRITON X-100, TERGITOL NP-9, TRICLOSAN or Chlorohexidine Digluconate (CHG) are not included at step B and titanium dioxide, essential oil package, farnesol and are included in step B.

### EXAMPLE 7

The following lotion formulation is similar to Example 6 in that it does not include any surfactants or the antimicrobial agents TRICLOSAN or Chlorohexidine Digluconate (CHG). In addition, this example includes titanium dioxide, essential oil package, farnesol and a “quaternary antibacterial package” (which includes Benzalkonium and Benzalthonium containing disinfecting agents). Examples of suitable Benzalkonium containing agents include, albeit not limited to, Benzalkonium chloride. An example of a Benzethonium containing agent includes, albeit not limited to, benzalthonium chloride. It should be noted that either one or both of the Benzalkonium and Benzalthonium containing disinfecting agents may be used in the instant formulation.

Excipients useful in the manufacture of this product were added in the following approximate amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | PROPYLENE GLYCOL U.S.P. | 0.5-5.0 |
| (5) | DOWCIDE-A | 0.0-2.0 |
| (6) | TRIETHANOLAMINE 85% N.F | 0.5-2.0 |
| (7) | BENZALKONIUM | 0.0-7.0 |
| (8) | BENZETHONIUM | 0.0-7.0 |
| (9) | ESSENTIAL OIL PACKAGE (#EOP-122) | 0.5-8.0 |
| (10) | ALPHA TOCOPHEROL | 0.5-8.0 |
| (11) | TITANIUM DIOXIDE | 0.05-7.0 |
| (12) | FARNESOL | 0.05-5.0 |

Wax phase

| | | |
|---|---|---|
| (13) | STEARIC ACID - TRIPLE PRESSED | 2.0-4.0 |
| (14) | CETYL ALCOHOL N.F. | 0.75-2.0 |
| (15) | ETHYLENE GLYCOL MONOSTEARATE | 0.25-1.5 |
| (16) | DIMETHICONE L-45-350 CSTKS | 0.5-3.5 |
| (17) | USP WHITE WAX | 0.1-1.0 |
| (18) | PARAGON MEPB | 0.5-6.0 |

The method for making the formulation of Example 7 follows the same steps A-D outlined in Example 1 above, the only difference is that TRITON X-100, TERGITOL NP-9, TRICLOSAN, and Chlorohexidine Digluconate (CHG) are not included at step B and titanium dioxide, essential oil package, farnesol, and either one or both the component quaternary antibacterial package (benzalkonium and benzethonium) are included in step B.

### EXAMPLE 8

The following formulation is encapsulated within a bead. The formulation is similar to Example 7 in that it does not include non-ionic surfactants TRITON X-100 and TERGITOL NP-9, but it may include the surfactant TWEEN-20 (although not required) or any other sorbitol-based surfactant or equivalent. Moreover, this example does not include titanium dioxide. As with the previous example the antimicrobial agents TRICLOSAN or Chlorohexidine Digluconate (CHG) are not used. However, the formulation does include essential oil package, farnesol and one or both of the components of the alternative quaternary antibacterial package. As with the previous example, either or both of the Benzalkonium and Benzalthonium containing disinfecting agents may be used in the instant formulation.

Excipients useful in the manufacture of this product were added in the following approximate amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | PROPYLENE GLYCOL U.S.P. | 0.5-5.0 |
| (5) | TWEEN-20 | 0.0-8.0 |
| (6) | DOWCIDE-A | 0.0-2.0 |
| (7) | TRIETHANOLAMINE 85% N.F | 0.5-2.0 |
| (8) | BENZALKONIUM | 0.0-7.0 |
| (9) | BENZETHONIUM | 0.0-7.0 |
| (10) | ESSENTIAL OIL PACKAGE (#EOP-122) | 0.5-8.0 |
| (11) | ALPHA TOCOPHEROL | 0.5-8.0 |
| (12) | FARNESOL | 0.05-5.0 |

Wax phase

| | | |
|---|---|---|
| (13) | STEARIC ACID - TRIPLE PRESSED | 2.0-4.0 |
| (14) | CETYL ALCOHOL N.F. | 0.75-2.0 |
| (15) | ETHYLENE GLYCOL MONOSTEARATE | 0.25-1.5 |
| (16) | DIMETHICONE L-45-350 CSTKS | 0.5-3.5 |
| (17) | USP WHITE WAX | 0.1-1.0 |
| (18) | PARAGON MEPB | 0.5-6.0 |

### EXAMPLE 9

The following formulation may include non-ionic surfactants TRITON X-100, TERGITOL NP-9, and surfactant TWEEN-20 or any other sorbitol based surfactant or equivalent if deemed necessary. This example also includes Titanium Dioxide, essential oil package, farnesol and one or more components of the alternative quaternary antibacterial package. In addition this formulation includes Alkali Soluble Emulsion polymers (ASE polymers) and solutions, synthesized from Acid and Acrylate co-monomers which are used as rheology modifiers. Examples of such rheology modifiers include, albeit not limited to, ACYULYN 33, ACUSOL 810-A, etc. The following formulation is used on a sanitizing wipe.

Excipients useful in the manufacture of this product were added in the following approximate amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | TRITON X-100 | 0.0-8.0 |
| (5) | PROPYLENE GLYCOL U.S.P. | 0.5-5.0 |
| (6) | TERGITOL NP-9 | 0.0-8.0 |
| (7) | TWEEN-20 | 0.0-8.0 |
| (8) | ASE POLYMERS | 0.01-5.0 |
| (9) | DOWCIDE-A | 0.0-2.0 |
| (10) | TRIETHANOLAMINE 85% N.F | 0.5-2.0 |
| (11) | BENZALKONIUM | 0.0-7.0 |
| (12) | BENZETHONIUM | 0.0-7.0 |
| (13) | ESSENTIAL OIL PACKAGE (#EOP-122) | 0.5-8.0 |
| (14) | ALPHA TOCOPHEROL | 0.5-8.0 |
| (15) | TITANIUM DIOXIDE | 0.02-7.0 |
| (16) | FARNESOL | 0.05-5.0 |

Wax phase

| | | |
|---|---|---|
| (17) | STEARIC ACID - TRIPLE PRESSED | 2.0-4.0 |
| (18) | CETYL ALCOHOL N.F. | 0.75-2.0 |
| (19) | ETHYLENE GLYCOL MONOSTEARATE | 0.25-1.5 |
| (20) | DIMETHICONE L-45-350 CSTKS | 0.5-3.5 |
| (21) | USP WHITE WAX | 0.1-1.0 |
| (22) | PARAGON MEPB | 0.5-6.0 |

The method for making the formulation of Example 9 follows the same steps A-D outlined in Example 1 above, the only difference is that TRICLOSAN or Chlorohexidine Digluconate (CHG) are not included at step B, however, TRITON X-100, TERGITOL NP-9 may be added at step B if desired. TWEEN-20, titanium dioxide, essential oil package, farnesol, one component of the quaternary antibacterial package (benzalkonium and benzethonium) and Alkali Soluble Emulsion polymers (ASE polymer) are included in step B.

### EXAMPLE 10

The following formulation may include non-ionic surfactants TRITON X-100, TERGITOL NP-9, and surfactant TWEEN-20 or any other sorbitol-based surfactant or equivalent if necessary. This example does include Titanium Dioxide, essential oil package, farnesol and at least one component of the quaternary antibacterial package (which includes Benzalkonium and Benzethonium containing disinfecting agents). In addition this formulation includes Alkali Soluble Emulsion polymers (ASE polymers) and solutions. The following formulation also includes a foaming surfactant (e.g., sodium laureth sulfate) for the foaming action of the hand wash. The hand wash may be used in any foam dispensing applicators or pumps.

Excipients useful in the manufacture of this product were added in the following amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | TRITON X-100 | 0.0-8.0 |
| (5) | PROPYLENE GLYCOL U.S.P. | 0.5-5.0 |
| (6) | TERGITOL NP-9 | 0.0-8.0 |
| (7) | FOAMING SURFACTANT | 0.2-50.0 |
| (8) | TWEEN-20 | 0.0-8.0 |
| (9) | DOWCIDE-A | 0.0-2.0 |
| (10) | TRIETHANOLAMINE 85% N.F | 0.5-2.0 |
| (11) | BENZALKONIUM | 0.0-7.0 |
| (12) | BENZETHONIUM | 0.0-7.0 |
| (13) | ESSENTIAL OIL PACKAGE (#EOP-122) | 0.5-8.0 |
| (14) | ALPHA TOCOPHEROL | 0.5-8.0 |
| (15) | TITANIUM DIOXIDE | 0.05-7.0 |
| (16) | FARNESOL | 0.05-5.0 |
| (17) | ASE POLYMERS | 0.01-5.0 |

Wax phase

| | | |
|---|---|---|
| (18) | STEARIC ACID - TRIPLE PRESSED | 2.0-4.0 |
| (19) | CETYL ALCOHOL N.F. | 0.75-2.0 |
| (20) | ETHYLENE GLYCOL MONOSTEARATE | 0.25-1.5 |
| (21) | DIMETHICONE L-45-350 CSTKS | 0.5-3.5 |
| (22) | USP WHITE WAX | 0.1-1.0 |
| (23) | PARAGON MEPB | 0.5-6.0 |

The method for making the formulation of Example 10 follows the same steps A-D outlined in Example 1 above, the only difference is that TRICLOSAN or Chlorohexidine Digluconate (CHG) are not included at step B; however, TRITON X-100, TERGITOL NP-9 may be added at step B if desired. Titanium dioxide, essential oil package, farnesol, quaternary antibacterial package (benzalkonium and/or benzethonium) and Alkali Soluble Emulsion polymers (ASE polymer) and a foaming agent surfactant are included in step B.

### EXAMPLE 11

The following formulation includes Alkali Soluble polymers (ASE polymers) and solutions. The following formulation is used in a sanitizing wipe.

Excipients useful in the manufacture of this product were added in the following approximate amounts:

| | |
|---|---|
| | WT/WT % |
| EXCIPIENT | RANGES |

Surfactant phase

| | | |
|---|---|---|
| (1) | DEIONIZED WATER | Q.S. |
| (2) | VERSENE-100 | 0.1-4.0 |
| (3) | CARBOPOL 934-P | 0.2-0.65 |
| (4) | TRITON X-100 | 1.0-8.0 |
| (5) | PROPYLENE GLYCOL U.S.P. | 0.5-5.0 |
| (6) | TERGITOL NP-9 | 1.0-8.0 |
| (7) | DOWCIDE-A | 0.0-2.0 |
| (8) | TRICLOSAN | 0.10-0.8 |
| (9) | TRIETHANOLAMINE 85% N.F | 0.5-2.0 |
| (10) | CHLOROHEXIDINE DIGLUCONATE 20% | 0.1-7.0 |
| (11) | ESSENTIAL OIL PACKAGE (#EOP-122) | 0.5-8.0 |
| (12) | ALPHA TOCOPHEROL | 0.5-8.0 |
| (13) | ASE POLYMERS | 0.01-5.0 |
| (14) | TITANIUM DIOXIDE | 0.02-7.0 |
| (15) | FARNESOL | 0.05-5.0 |

Wax phase

| | | |
|---|---|---|
| (16) | STEARIC ACID - TRIPLE PRESSED | 2.0-4.0 |
| (17) | CETYL ALCOHOL N.F. | 0.75-2.0 |
| (18) | ETHYLENE GLYCOL MONOSTEARATE | 0.25-1.5 |
| (19) | DIMETHICONE L-45-350 CSTKS | 0.5-3.5 |
| (20) | USP WHITE WAX | 0.1-1.0 |
| (21) | PARAGON MEPB | 0.5-6.0 |

The method for making the formulation of Example 11 follows the same steps A-D outlined in Example 1 above, the only difference is that titanium dioxide, essential oil package, farnesol, quaternary antibacterial package (benzalkonium and benzethonium) and Alkali Soluble Emulsion polymers (ASE polymer) are included in step B.

In examples 7, 8, 9, and 10, outlined above, the agents of the quaternary antibacterial package may be replaced with the Chloroxylenol antimicrobial agent in the range of about 0.002 to about 4.0 wt/wt %; that is, a formulation which includes Chloroxylenol will not contain either Benzalkonium or Benzalthonium. DOWCIDE-A is a preservative which potentiates the active antimicrobial agent(s) (TRICLOSAN, chlorohexidine digluconate, quaternary antibacterial package, chloroxylenol) in any of the aforementioned formulations; however, its inclusion in any of the formulation of examples is not required. Moreover, any one of examples above may include least one fragrance therein in the range of about 0.5 to about 1.0 wt/wt %.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims.

Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the invention.

What is claimed is:

1. A grain free aqueous antimicrobial sanitizing composition, characterized by enhanced antimicrobial and skin protective properties comprising, in combination:
a surfactant phase including about 0.1-4.0% by weight of a chelating agent; about 0.2-0.65% by weight of an acrylic acid polymer; about 1.0-8.0% by weight of an octyl phenoxypolyethoxy non-ionic surfactant; about 0.5-5.0% by weight of propylene glycol; about 1.0-8.0% by weight of a nonylphenol polyethylene glycol ether non-ionic surfactant; about 0.0-2.0% by weight of a phenate antimicrobial; about 0.1-0.8% by weight of a 5-chloro-2-(2,4-dichlorophenoxy)-phenol; about 0.5-2.0% by weight of triethanolamine 85% N.F.; about 0.1-7.0% by weight of chlorhexidene gluconate 20%; about 0.5-8.0% by eight of an alpha tocopherol; and deionized water in an amount to make 100% by weight of said surfactant phase;
a wax phase including about 2.0-4.0% by weight triple pressed stearic acid; about 0.75-2.0% by weight cetyl alcohol N.F.; about 0.25-1.5 ethylene glycol monostearate; about 0.5-3.5% by weight dimethicone; about 0.1-1.0% by weight USP White Wax; and
about 0.5-6.0% by weight of a mixture of Methyl, Ethyl, Propyl, and Butyl Parabenzene in a Phenoxy Ethanol solvent;
wherein contact with the skin results in destruction of microbial contaminants and simultaneous formation of a hydrophobic skin protective barrier layer.

2. The composition of claim 1 further including about 0.05-7.0% by weight titanium dioxide, about 0.05-5.0% by weight farnesol and about 0.5-8.0% by weight essential oils, added to said surfactant phase.

3. A grain free aqueous antimicrobial sanitizing composition, characterized by enhanced antimicrobial and skin protective properties comprising, in combination:
a surfactant phase including about 0.1-4.0% by weight of a chelating agent; about 0.2-0.65% by weight of an acrylic acid polymer; about 0.5-5.0% by weight of propylene glycol; about 0.0-2.0% by weight of a phenate antimicrobial; about 0.1-0.8% by weight of a 5-chloro-2-(2,4-dichlorophenoxy)-phenol; about 0.5-2.0% by weight of triethanolamine 85% N.F.; about 0.1-7.0% by weight of chlorhexidene gluconate 20%; about 0.5-8.0% by eight of an alpha tocopherol; about 0.05-7.0% by weight titanium dioxide, about 0.05-5.0% by weight farnesol and about 0.5-8.0% by weight essential oils, and deionized water in an amount to make 100% by weight of said surfactant phase;
a wax phase including about 2.0-4.0% by weight triple pressed stearic acid; about 0.75-2.0% by weight cetyl alcohol N.F.; about 0.25-1.5 ethylene glycol monostearate; about 0.5-3.5% by weight dimethicone; about 0.1-1.0% by weight USP White Wax; and
about 0.5-6.0% by weight of a mixture of Methyl, Ethyl, Propyl, and Butyl Parabenzene in a Phenoxy Ethanol solvent;
wherein contact with the skin results in destruction of microbial contaminants and simultaneous formation of a hydrophobic skin protective barrier layer.

4. The composition of claim 3 further including about 0.5-8.0% by weight of a sorbitol based surfactant in the surfactant phase.

5. A grain free aqueous antimicrobial sanitizing composition, characterized by enhanced antimicrobial and skin protective properties comprising, in combination:
a surfactant phase including about 0.1-4.0% by weight of a chelating agent; about 0.2-0.65% by weight of an acrylic acid polymer; about 0.5-5.0% by weight of propylene glycol; about 0.0-2.0% by weight of a phenate antimicrobial; about 0.5-2.0% by weight of triethanolamine 85% N.F; about 0.5-8.0% by eight of an alpha tocopherol; about 0.05-7.0% by weight titanium dioxide, about 0.05-5.0% by weight farnesol and about 0.5-8.0% by weight essential oils, about 0.5-8.0% by weight of a sorbitol based surfactant; and deionized water in an amount to make 100% by weight of said surfactant phase;
a wax phase including about 2.0-4.0% by weight triple pressed stearic acid; about 0.75-2.0% by weight cetyl alcohol N.F.; about 0.25-1.5 ethylene glycol monostearate; about 0.5-3.5% by weight dimethicone; about 0.1-1.0% by weight USP White Wax; and
about 0.5-6.0% by weight of a mixture of Methyl, Ethyl, Propyl, and Butyl Parabenzene in a Phenoxy Ethanol solvent;
wherein contact with the skin results in destruction of microbial contaminants and simultaneous formation of a hydrophobic skin protective barrier layer.

6. A grain free aqueous antimicrobial sanitizing composition, characterized by enhanced antimicrobial and skin protective properties comprising, in combination:
a surfactant phase including about 0.1-4.0% by weight of a chelating agent; about 0.2-0.65% by weight of an acrylic acid polymer; about 0.5-5.0% by weight of propylene glycol; about 0.0-2.0% by weight of a phenate antimicrobial; about 0.5-2.0% by weight of triethanolamine 85% N.F; about 0.5-8.0% by eight of an alpha tocopherol; about 0.05-7.0% by weight titanium dioxide, about 0.05-5.0% by weight farnesol and about 0.5-8.0% by weight essential oils; and deionized water in an amount to make 100% by weight of said surfactant phase;
a wax phase including about 2.0-4.0% by weight triple pressed stearic acid; about 0.75-2.0% by weight cetyl alcohol N.F.; about 0.25-1.5 ethylene glycol monostearate; about 0.5-3.5% by weight dimethicone; about 0.1-1.0% by weight USP White Wax; and
about 0.5-6.0% by weight of a mixture of Methyl, Ethyl, Propyl, and Butyl Parabenzene in a Phenoxy Ethanol solvent;
wherein contact with the skin results in destruction of microbial contaminants and simultaneous formation of a hydrophobic skin protective barrier layer.

7. A grain free aqueous antimicrobial sanitizing composition, characterized by enhanced antimicrobial and skin protective properties comprising, in combination:
a surfactant phase including about 0.1-4.0% by weight of a chelating agent; about 0.2-0.65% by weight of an acrylic acid polymer; about 0.5-5.0% by weight of propylene glycol; about 0.0-2.0% by weight of a phenate antimicrobial; about 0.5-2.0% by weight of triethanolamine 85% N.F; about 0.5-8.0% by eight of an alpha tocopherol; about 0.05-7.0% by weight titanium dioxide, about 0.05-5.0% by weight farnesol and about 0.5-8.0% by weight essential oils; up to about 7.0% by weight of a benzalkonium disinfecting agent and up to 7.0% of a benzethonium disinfecting agent; and deionized water in an amount to make 100% by weight of said surfactant phase;
a wax phase including about 2.0-4.0% by weight triple pressed stearic acid; about 0.75-2.0% by weight cetyl alcohol N.F.; about 0.25-1.5 ethylene glycol monostearate; about 0.5-3.5% by weight dimethicone; about 0.1-1.0% by weight USP White Wax; and
about 0.5-6.0% by weight of a mixture of Methyl, Ethyl, Propyl, and Butyl Parabenzene in a Phenoxy Ethanol solvent;
wherein contact with the skin results in destruction of microbial contaminants and simultaneous formation of a hydrophobic skin protective barrier layer.

8. A bead encapsulated grain free aqueous antimicrobial sanitizing composition, characterized by enhanced antimicrobial and skin protective properties comprising, in combination:
a surfactant phase including about 0.1-4.0% by weight of a chelating agent; about 0.2-0.65% by weight of an acrylic acid polymer; about 0.5-5.0% by weight of propylene glycol; about 0.0-2.0% by weight of a phenate antimicrobial; about 0.5-2.0% by weight of triethanolamine 85% N.F; about 0.5-8.0% by eight of an alpha tocopherol; about 0.05-5.0% by weight farnesol and about 0.5-8.0% by weight essential oils; up to about 7.0% by weight of a benzalkonium disinfecting agent and up to 7.0% of a benzethonium disinfecting agent; and deionized water in an amount to make 100% by weight of said surfactant phase;
a wax phase including about 2.0-4.0% by weight triple pressed stearic acid; about 0.75-2.0% by weight cetyl alcohol N.F.; about 0.25-1.5 ethylene glycol monostearate; about 0.5-3.5% by weight dimethicone; about 0.1-1.0% by weight USP White Wax; and
about 0.5-6.0% by weight of a mixture of Methyl, Ethyl, Propyl, and Butyl Parabenzene in a Phenoxy Ethanol solvent;
wherein contact with the skin results in destruction of microbial contaminants and simultaneous formation of a hydrophobic skin protective barrier layer.

9. The composition of claim 7 further including 0.01-5.0% by weight of at least one alkali soluble emulsion modifier in an amount effective to act as a rheology modifier.

10. A grain free aqueous antimicrobial sanitizing composition, characterized by enhanced antimicrobial and skin protective properties comprising, in combination:
a surfactant phase including about 0.1-4.0% by weight of a chelating agent; about 0.2-0.65% by weight of an acrylic acid polymer; up to about 8.0% by weight of an octyl phenoxypolyethoxy non-ionic surfactant; about 0.5-5.0% by weight of propylene glycol; up to about 8.0% by weight of a nonylphenol polyethylene glycol ether non-ionic surfactant; from 0.2-50% by weight of a foaming surfactant, about 0.0-2.0% by weight of a phenate antimicrobial; about 0.5-2.0% by weight of triethanolamine 85% N.F; about 0.5-8.0% by eight of an alpha tocopherol; about 0.05-5.0% by weight farnesol; 0.01-5.0% by weight of at least one alkali soluble emulsion modifier; about 0.5-8.0% by weight essential oils; up to about 7.0% by weight of a benzalkonium disinfecting agent and up to 7.0% of a benzethonium disinfecting agent; and deionized water in an amount to make 100% by weight of said surfactant phase;
a wax phase including about 2.0-4.0% by weight triple pressed stearic acid; about 0.75-2.0% by weight cetyl alcohol N.F.; about 0.25-1.5 ethylene glycol monostearate; about 0.5-3.5% by weight dimethicone; about 0.1-1.0% by weight USP White Wax; and
about 0.5-6.0% by weight of a mixture of Methyl, Ethyl, Propyl, and Butyl Parabenzene in a Phenoxy Ethanol solvent;
wherein contact with the skin results in destruction of microbial contaminants and simultaneous formation of a hydrophobic skin protective barrier layer.

11. A grain free aqueous antimicrobial sanitizing composition, characterized by enhanced antimicrobial and skin protective properties comprising, in combination:
a surfactant phase including about 0.1-4.0% by weight of a chelating agent; about 0.2-0.65% by weight of an acrylic acid polymer; up to about 8.0% by weight of an octyl phenoxypolyethoxy non-ionic surfactant; about 0.5-5.0% by weight of propylene glycol; up to about 8.0% by weight of a nonylphenol polyethylene glycol ether non-ionic surfactant; from 0.2-50% by weight of a foaming surfactant, about 0.0-2.0% by weight of a phenate antimicrobial; about 0.5-2.0% by weight of triethanolamine 85% N.F; about 0.5-8.0% by eight of an alpha tocopherol; about 0.05-5.0% by weight farnesol; 0.01-5.0% by weight of at least one alkali soluble emulsion modifier; about 0.5-8.0% by weight essential oils; about 002-4.0% by weight of a chloroxylenol and deionized water in an amount to make 100% by weight of said surfactant phase;
a wax phase including about 2.0-4.0% by weight triple pressed stearic acid; about 0.75-2.0% by weight cetyl alcohol N.F.; about 0.25-1.5 ethylene glycol monostearate; about 0.5-3.5% by weight dimethicone; about 0.1-1.0% by weight USP White Wax; and
about 0.5-6.0% by weight of a mixture of Methyl, Ethyl, Propyl, and Butyl Parabenzene in a Phenoxy Ethanol solvent;
wherein contact with the skin results in destruction of microbial contaminants and simultaneous formation of a hydrophobic skin protective barrier layer.

12. An antimicrobial sanitizing product including any one of the compositions of claim 1, 2, 3, 4, 5, 6, 7, 10 and 11 provided in the form of a lotion, a wipe, a soap, an encapsulated bead, or a skin cleansing polymer/emulsion.
